(19) United States
(12) Patent Application Publication
(10) Pub. No.: US 2005/0026274 A1
(43) Pub. Date: Feb. 3, 2005

(54) Device and method for selecting locomotive biological species, in particularly sperm cells

(76) Inventor: Josef Zech, Innsbruck (AT)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/491,309

(22) PCT Filed: Oct. 1, 2002
(86) PCT No.: PCT/EP02/11019

(30) Foreign Application Priority Data
Oct. 5, 2001 (AT) A 1574/01

(51) Int. Cl. C12M 1/34

(52) U.S. Cl. 435/287.1

(57) ABSTRACT

The present invention provides an apparatus for selecting motile biological species, in particular sperm, comprising a first chamber for receiving a medium containing the species to be selected and, separate therefrom, a second chamber for receiving the selected species in a further medium, and characterized in that it comprises a bridge element mountable on the chambers and having at least one flat channel formed by limiting walls which, when the bridge element is mounted, extends from the first chamber to the second chamber, has an opening in the area of the first chamber and the second chamber in each case, and is fillable with a medium in which the motile biological species can move, such that the medium in the first chamber is connected with that in the second chamber.

The present invention relates to an apparatus for selecting motile biological species, in particular for selecting sperm, comprising a first chamber for receiving a medium containing the biological species to be selected and, separate therefrom, a second chamber for receiving the selected species in a further medium.

Further, the present invention relates to a method for selecting motile biological species, in particular sperm, and the use of the inventive apparatus for selecting motile biological species, in particular sperm.

In extracorporeal fertilization, the first step is usually recovery/selection of sperm from seminal fluid (ejaculate). In a second step the selected sperm are then contacted with one or more egg cells in vitro or in vivo.

The selection and separation of sperm from seminal fluid was hitherto done predominantly by centrifugation. However, this method has the disadvantage firstly that the sperm are greatly loaded mechanically and thus damage done to (healthy) sperm. Secondly, motile sperm can be selected and separated from sperm with little or no motility or that are dead only in limited fashion by centrifugation.

Further, EP 0 683 645 discloses an apparatus for selecting sperm from seminal fluid comprising two chambers, the first chamber being filled with seminal fluid and the second chamber with a nutrient solution for receiving selected sperm. Between the chambers a liquid bridge is produced by a capillary tube spanning the dividing wall of the chambers or by loosely mounting a bridge member. Selection of sperm is effected in this apparatus in such a way that motile sperm can pass from the first chamber to the second via the liquid bridge. However, this known apparatus has the disadvantages that, firstly, the selection of sperm takes place very slowly with use of a capillary tube while, secondly, the liquid bridge is not formed in predefined, reproducible fashion with use of a loosely mounted bridge member, so that selection of sperm is frequently again effected very slowly, if at all.

It is a problem of the present invention to provide an apparatus for selecting motile biological species, in particular sperm, which does not mechanically load the motile biological species and permits for example motile sperm to be separated from sperm with little or no motility.

Further, it is a problem of the present invention to provide such an apparatus by which selection of the motile biological species is effected as rapidly as possible and which guarantees simple and safe handling by laboratory staff such as medical laboratory assistants.

The present invention is based on the finding that such an apparatus must comprise a first chamber for receiving a medium containing the biological species to be selected and a second chamber for receiving a further medium for the selected species and have at least one flat channel that is fillable with a medium in which the species can move and in the filled state of the apparatus interconnects the media located in the first and second chambers, said channel being formed in defined fashion independently of the handling of the apparatus by the user.

The present invention therefore provides, in a first embodiment, an apparatus for selecting motile biological species, in particular sperm, comprising a first chamber for receiving a medium containing the species to be selected and, separate therefrom, a second chamber for receiving the selected species in a further medium, and characterized in that it comprises a bridge element mountable on the chambers and having at least one flat channel formed by limiting walls which, when the bridge element is mounted, extends from the first chamber to the second chamber, has an opening in the area of the first chamber and the second chamber in each case, and is fillable with a medium in which the motile biological species can move, in such a way that the medium in the first chamber is connected with that in the second chamber.

“Motile biological species” refer according to the present invention to those organic species such as microorganisms or, in particular, sperm, that can move independently within a medium, such as a liquid.

The term “flat channel” refers according to the present invention to a hollow space having a smaller extension in one dimension than in the other two dimensions. Preferably, the extension of the hollow space in the other two dimensions is a multiple of the extension in the first dimension.

The term “medium” refers according to the invention to any kind of matter in which the species to be selected can move. Preferred media are fluid media such as liquids and gels. Said fluid media and gels can also contain further substances such as nutrients for the biological species and electrolytes.

For example, in the selection of sperm the medium containing the species to be selected can be seminal fluid, and the medium receiving the selected sperm a nutrient solution. In this case, nutrient solution is likewise the preferred medium for filling the channel.

The handling of the apparatus for example for selecting sperm from seminal fluid consists in the user first filling seminal fluid into a first chamber. Subsequently, the bridge element is mounted on the chambers, and the second chamber and the channel or channels filled with nutrient solution, whereby the seminal fluid in the first chamber is preferably also covered with a layer of nutrient solution. In the filled state, a liquid bridge thus arises between the first and second chambers, in which there is no convection of the liquid contained therein. Motile sperm can then travel via said bridge from the seminal fluid to the nutrient solution of the second chamber. Thus, sperm are not exposed to mechanical load during selection and motile sperm are separated from nonmotile. The selected sperm contained in the second chamber can then be removed for example with a pipette for insemination, cryoconservation or diagnostic purposes for extracorporeal fertilization. It is also possible to insert one or more egg cells directly into the second chamber for fertilization.

In the inventive apparatus of this first embodiment, the mountable bridge element itself has at least one flat channel that already has a defined form, i.e. it has fixed dimensions predefined by the fixed arrangement of the limiting walls. This means that in the filled state the connection of the chambers by the medium present in the channel can form in optimal fashion after the bridge element is mounted on the chambers. This comes about independently of the handling of the apparatus by laboratory staff. It is thus guaranteed that the selection of the motile biological species, in particular sperm, is always effected in reproducible, rapid fashion.

A further advantage of the inventive apparatus is that the medium in the channels of the apparatus suffices, i.e. is not subjected to convection, so that the species to be selected are selected only on the basis of their motility and thus for example the sperm most active for fertilization can be obtained.

In the selection of sperm from seminal fluid performed with the inventive apparatus, a sufficient quantity of motile sperm, which are enough for example for extracorporeal fertilization, can already be selected to the second chamber of the apparatus after about 15 to 30 minutes.

In a second embodiment of the present invention, the apparatus for selecting motile biological species, in particular sperm, comprises a first chamber for receiving a medium containing the species to be selected and, separate therefrom, a second chamber for receiving the selected species in a further medium, the walls of the first chamber and second chamber being at least partly interconnected and a bridge element being mountable on the chambers such that the connected walls and one side of the bridge element each form a limiting wall of a flat channel having an opening in the area of the first chamber and the second chamber in each case and fillable with a medium in which the motile biological species can move, such that the medium in the first chamber is connected with that in the second chamber and characterized in that at least one adjusting element is provided such that the channel is always formed in the same, defined fashion when the bridge element is mounted.

In this second embodiment of the inventive apparatus, a flat channel is reliably always formed in predefined, reproducible fashion when the bridge element is mounted on the connecting wall of the chambers by the user, i.e. the channel has the dimensions predefined by the arrangement of the limiting walls in connection with the adjusting element independently of the handling by the user. The adjusting element makes it possible to give the channel formed an optimal shape for formation of the liquid bridge. Thus, this embodiment of the present invention also make it possible to attain the advantages stated for the first embodiment and therefore achieve rapid and reproducible selection of motile biological species, independently of the handling of the apparatus by the user.

Preferably, the inventive apparatus of the first embodiment is formed so that the walls of the first chamber and second chamber are at least partly interconnected and the bridge element is mountable on the chambers such that the connected walls and one side of the bridge element each form a limiting wall of a flat channel having an opening in the area of the first chamber and the second chamber in each case and being fillable with a medium in which the motile biological species can move, such that the medium in the first chamber is connected with that in the second chamber.

In this preferred embodiment, at least two flat channels in which a connection of the first and second chambers can form by a medium are present between the first and second chambers in the mounted state of the bridge element.

Further preferably, an adjusting element is provided in the apparatus of this embodiment such that the channel formed by the connected walls of the chambers and one side of the bridge element as limiting walls is always formed in the same, defined fashion when the bridge element is mounted.

This preferred embodiment of the inventive apparatus, which combines the features of the above-described first and second embodiments, guarantees that the second channel formed by the user mounting the bridge element on the chambers is also always formed in predefined, reproducible fashion, so that the selection of the motile biological species can be effected even more effectively and rapidly.

The preferred embodiments described in the following relate to all embodiments of the inventive apparatus described above.

In a preferred embodiment of the inventive apparatus, the bridge element mountable on the chambers has at least two, further preferably at least three, and even more preferably, at least four, flat channels formed by limiting walls, which, when the bridge element is mounted, extend from the first chamber to the second chamber, have an opening in the area of the first chamber and the second chamber in each case, and are fillable with a medium in which the motile biological species can move, such that the medium in the first chamber is connected with that in the second chamber.

These preferred embodiments of the inventive apparatus guarantee, firstly, that the selection of motile biological species takes place even more efficiently and rapidly and, secondly, that selection can nevertheless take place if one channel fails to work for example due to soiling or incomplete formation of the bridging of the two chambers by the medium present in the channel, e.g. due to air bubbles in a liquid medium.

In a further preferred embodiment of the inventive apparatus, the distance between the opposing limiting walls forming a channel, or parts of said walls, is predefined such that capillary forces act on the medium with which the channel is filled, so that upon contact with the medium the channel independently becomes saturated at least partly, preferably completely.

This distance causing the capillary forces corresponds to the abovementioned first dimension of the flat channels. This embodiment facilitates the filling of the channel with medium.

Further preferably, the inventive apparatus according to one of the above embodiments is formed such that the opposing limiting walls forming a channel, or parts of limiting walls, have a maximum distance apart of 1.0 millimeters, preferably 0.5 millimeters.

Further preferably, the opposing limiting walls forming a channel, or parts of limiting walls, have a minimum distance apart of 0.1 millimeters, preferably 0.2 millimeters.

Especially preferably, opposing limiting walls have a distance apart in the range of 0.3 to 0.4 millimeters.

The stated preferred distances between limiting walls correspond to the abovementioned first dimension of the channels. The stated preferred values ensure an optimal formation preferably of a liquid bridge of aqueous medium or nutrient solution in the channels through the capillary action of the channels.

Further preferably, the opposing limiting walls of a channel extend parallel to each other in the inventive apparatus.

In a further preferred embodiment, the channels of the inventive apparatus are formed so that a pair of channels at least partly shares a limiting wall. Further preferably, a pair of channels of the apparatus shares a total limiting wall.

This permits an especially simple and material-saving design of the inventive apparatus.

Preferably, the limiting walls of the channels and thus also the channels of the apparatus themselves have a U-shaped cross section.

Further preferably, the channels of the inventive apparatus protrude farther into the second chamber than into the first chamber. This simplifies the handling of the apparatus.

In a further preferred embodiment, at least one channel of the apparatus protrudes a different distance into the first chamber in comparison with the other channels. Further preferably, all channels that the apparatus has protrude different distances into the first chamber.

The different insertion depths of the channels into the first chamber facilitate the exit of especially a medium with higher surface tension from the channels through improved drop formation, thereby facilitating the filling of the channels. This applies for example to nutrient fluid for sperm, which has a considerably higher surface tension in comparison with water.

Further preferably, the inventive apparatus is so constituted that one of the chambers is of annular form and encloses the other, second chamber.

Preferably, the chambers and the bridge element of the inventive apparatus are in rotationally symmetric arrangement.

Further preferably, the two chambers of the apparatus are part of a chamber element made of one piece.

The inventive apparatus can be constructed of materials such as glass or plastic. But preferably plastic, further preferably transparent plastic, is used.

The use of plastic as the material for the inventive apparatus has the advantage that the apparatus can be produced in simple and thus economically advantageous fashion. Due to this economic advantage, it is possible for the user to dispose of the inventive apparatus after one-time use, thereby eliminating hygienically dubious reuse. Secondly, the inventive apparatus can be sterilized in simple fashion, for example by electron bombardment.

The inventive apparatus can be placed in an outer container preferably comprising a bottom part and a cover, for example for transport or storage.

Further preferably, the container is formed such that the inventive apparatus is fixed in place therein in the assembled state.

The present invention further provides a method for selecting motile biological species, in particular sperm, wherein the species to be selected are placed in a medium in a first chamber of an apparatus and the selected species are received in a further medium in a second chamber of the apparatus, characterized in that the media in the chambers of the apparatus are connected via a medium present in a flat channel formed by limiting walls in predefined fashion, whereby the motile species can pass from the first chamber to the second through their own motion via the medium present in the channel and are thus selected.

In preferred embodiments of the inventive method, an apparatus according to one of the above-described embodiments is used.

Further, the present invention also relates to the use of an apparatus according to one of the above-described embodiments for selecting motile biological species contained in a liquid, in particular for selecting sperm contained in seminal fluid.

In the following an embodiment of the inventive apparatus will be described with reference to the enclosed figures, in which:

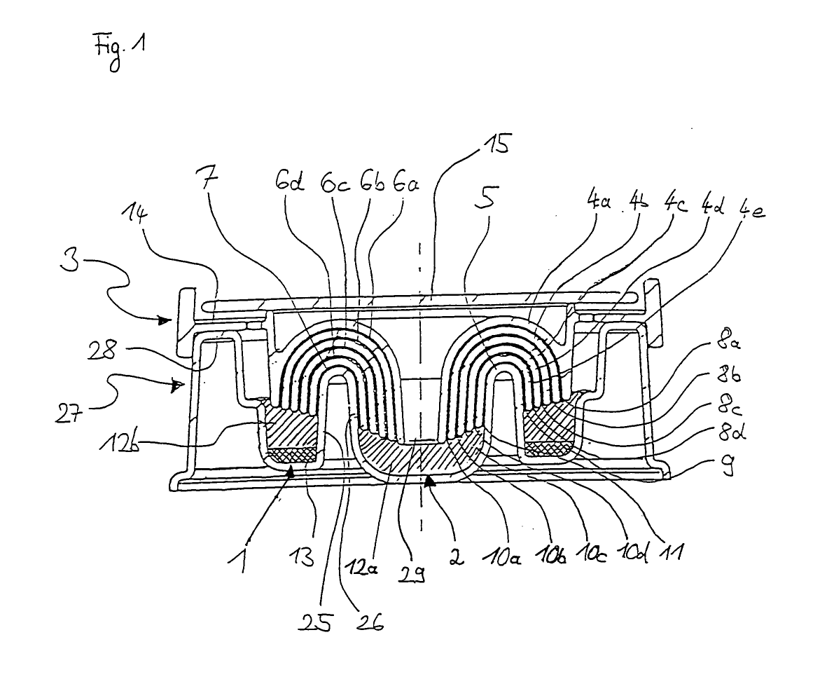

FIG. 1 shows a cross section of the inventive apparatus in the assembled state, and

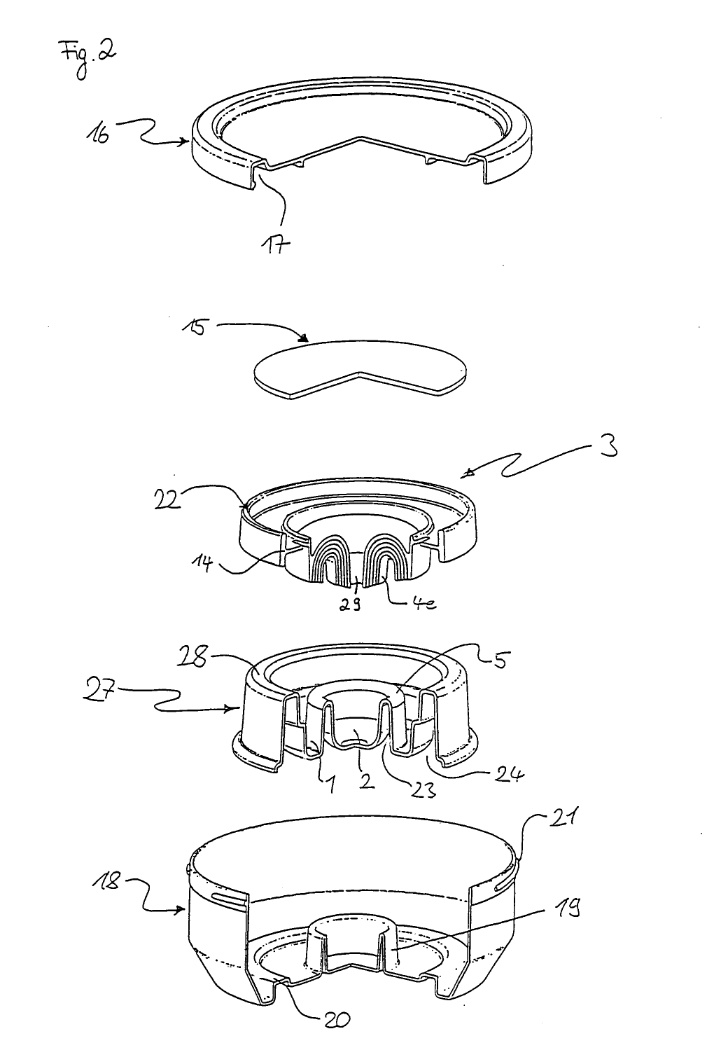

FIG. 2 shows partly sectional oblique views of the inventive apparatus and a container therefor.

The inventive apparatus of this embodiment is formed by a bridge element (3) comprising four channels (6a-d) of predefined form, and by a chamber element (27) having an annular first chamber (1) for receiving for example seminal fluid (13) and a second chamber (2) enclosed completely by said annular chamber (12a), for receiving nutrient fluid.

The bridge element (3) has altogether five limiting walls (4a-e) which have a U-shaped cross section and annularly span the annular connection (5) of the inner outside wall of the annular first chamber (25) and the outer wall of the second, central chamber (26). The limiting walls (4a-e) are all disposed parallel to each other and have a distance apart of about 0.3 millimeters. The parallel alignment of the limiting surfaces (4a-e) and the precise distance apart can be obtained for example by punctiform connections between the limiting walls (4a-e).

The limiting walls (4a-e) form the four channels (6a-d) which are all part of the bridge element. A further channel (7) is formed by the inner surface of the limiting wall (4e) of the bridge element (3) and the outer surface of the connected walls of the chambers (5) when the bridge element (3) is mounted on the chamber element (27).

The adjusting element (14) present on the bridge element (3), which is executed as a T-shaped ring and can be mounted in shape-mated fashion on the upper circumferential wall (28) of the chamber element (27), guarantees that the bridge element (3) is always mountable on the chamber element (27) by the user in precisely defined and centered fashion, thereby guaranteeing the constant distance between the straight parts of the limiting walls (4e) and (5) of the channel (7). Further, the adjusting element (14) guarantees that the bridge element (3) is also always mounted on the chamber element (27) at a defined, predetermined height, so that the predetermined distance between the limiting walls likewise comes about in the turning area of the limiting walls (4e) and (5). The annular formation of the adjusting element (14) also permits the first, annular chamber (1) to be completely covered in the assembled state of the apparatus. This avoids soiling of the seminal fluid.

Further, a cover (15) is present which can be mounted on the bridge element (3) after assembly and filling of the apparatus. This avoids soiling of the nutrient solution present in the second chamber (2).

For carrying out the selection of sperm from seminal fluid, seminal fluid with sperm (13) to be selected is filled into the annular chamber (1) in such a way that the seminal fluid does not yet reach the openings (8a-d, 9) of the channels on the side of the annular chamber (1) in the assembled state of the apparatus. Subsequently, the bridge element (3) is mounted on the chambers (1, 2).

Nutrient fluid (12a) is then filled into the central chamber (2) through the central opening (29) in such a way that the channels (6a-d, 7) become completely saturated with nutrient fluid due to the higher liquid level in the second chamber (2) or the pressure exerted on the liquid during filling, for example with a pipette, and the capillary forces acting in the channels, and the seminal fluid (13) of the annular chamber (1) is covered with a layer of nutrient fluid (12b).

As soon as the covering of seminal fluid (13) with a layer of nutrient fluid (12b) is over, the nutrient fluid rests in the channels, i.e. is not subjected to convection, and motile sperm begin to travel into the nutrient solution (12a) of the central chamber (2) due to their own motion through the filled channels (6a-d, 7) acting as liquid bridges.

The limiting walls (4a-e) of the bridge element (3) are formed such that the opening (8a) of the channel (6a) has the smallest insertion depth into the annular first chamber (1) and the insertion depth of the openings (8b, c, d, 9) of the channels (6b, c, d, 7) into the first chamber (1) continually increases. Conversely, the insertion depth of the openings (10a-d, 11) of the channels (6a-d, 7) into the second, central chamber (2) continually decreases.

To protect the inventive apparatus from contamination, for example during transport, a container (18) with a matching cover (16) is provided, said container having, for fixing the apparatus in place, annular lands (19) and (2) capable of engaging corresponding gaps (23) and (24) of the chamber element (27). The cover (16) has an annular gap (17) that is mountable in shape-mated fashion on the profiled annular edge (22) of the centering element (14) connected with the bridge element (3). The cover can be closed for example by a screw closure (21).

1. An apparatus for selecting motile biological species, in particular sperm, comprising a first chamber for receiving a medium containing the species to be selected and, separate therefrom, a second chamber for receiving the selected species in a further medium, characterized in that it comprises a bridge element mountable on the chambers and having at least one flat channel formed by limiting walls which, when the bridge element is mounted, extends from the first chamber to the second chamber has an opening in the area of the first chamber and the second chamber in each case, and is fillable with a medium in which the motile biological species can move, such that the medium in the first chamber is connected with that in the second chamber.

2. An apparatus for selecting motile biological species, in particular sperm, comprising a first chamber for receiving a medium containing the species to be selected and, separate therefrom, a second chamber for receiving the selected species in a further medium, the walls of the first chamber and the second chamber being at lest partly interconnected and a bridge element being mountable on the chambers such that the connected walls and one side of the bridge element each form a limiting wall of a flat channel which has an opening in the area of the first chamber and the second chamber in each case and is fillable with a medium in which the motile biological species can move, such that the medium in the first chamber is connected with that in the second chamber, characterized in that at least one adjusting element is provided such that the channel is always formed in the same, defined fashion when the bridge element is mounted.

3. An apparatus according to claim 1, characterized in that the walls of the first chamber and the second chamber are at least partly interconnected and the bridge element is mountable on the chambers such that the connected walls and one side of the bridge element each form a limiting wall of a flat channel which has an opening in the area of the first chamber and the second chamber in each case and is fillable with a medium in which the motile biological species can move, such that the medium in the first chamber is connected with that in the second chamber.

4. An apparatus according to claim 3, characterized in that at least one adjusting element is provided such that channel formed by the connected walls of the chambers and one side of the bridge element as limiting walls is always formed in the same, defined fashion when the bridge element is mounted.

5. An apparatus according to claims 1, characterized in that the distance between the opposing limiting walls forming a channel, or parts of said limiting walls, is predefined such that capillary forces act on the medium with which the channel is filled, so that the channel independently becomes saturated completely or partly upon contact with the medium.

6. An apparatus according to claims 1, characterized in that the opposing limiting walls forming a channel, or parts of limiting walls, have a maximum distance apart of 1.0 millimeters, preferably 0.5 millimeters, and a minimum distance apart of 0.1 millimeters, preferably 0.2 millimeters.

7. An apparatus according to claims 1, characterized in that the opposing limiting walls of a channel extend parallel to each other.

8. An apparatus according to claims 1, characterized in that a pair of channels at least partly shares a limiting wall.

9. An apparatus according to claims 1, characterized in that the limiting walls of the channels have a U-shaped cross section.

10. An apparatus according to claims 1, characterized in that one of the chambers is of annular form and encloses the other chamber.

11. A method for selecting motile biological species, in particular sperm, wherein the species to be selected are placed in a medium in a first chamber of an apparatus and the selected species are received in a second chamber of the apparatus in a further medium, characterized in that the media in the chambers of the apparatus are connected via a medium present in a flat channel formed by limiting walls in predefined fashion, whereby the motile species can pass from the first chamber to the second through their own motion via the medium present in the channel and are thus selected.

12. A method according to claim 11, characterized in that an apparatus according to claims 1 is used.

13. Use of an apparatus according to claims 1 for selecting motile biological species, in particular for selecting sperm.

14. An apparatus according to claim 2, characterized in that the distance between the opposing limiting walls forming a channel, or parts of said limiting walls, is predefined such that capillary forces act on the medium with which the channel is filled, so that the channel independently becomes saturated completely or partly upon contact with the medium.

15. An apparatus according to claim 2, characterized in that the opposing limiting walls forming a channel, or parts of limiting walls, have a maximum distance apart of 1.0 millimeters, preferably 0.5 millimeters, and a minimum distance apart of 0.1 millimeters, preferably 0.2 millimeters.

16. An apparatus according to claim 2, characterized in that the opposing limiting walls of a channel extend parallel to each other.

17. An apparatus according to claim 2, characterized in that a pair of channels at least partly shares a limiting wall.

18. An apparatus according to claim 2, characterized in that the limiting walls of the channels have a U-shaped cross section.

19. An apparatus according to claim 2, characterized in that one of the chambers is of annular form and encloses the other chamber.
